(19) United States
(12) Patent Application Publication
(10) Pub. No.: US 2022/0308032 A1
(43) Pub. Date: Sep. 29, 2022

(54) Cannabis Proficiency Testing Material

(71) Applicant: Research Triangle Institute, Research Triangle Park, NC (US)

(72) Inventors: E. Dale Hart, Cary, NC (US); Nichole D. Bynum, Cary, NC (US); Lisa C. Greene, Durham, NC (US); Katherine M. Bollinger, Raleigh, NC (US); Megan A. Grabenauer, Los Osos, CA (US); Andrea C. McWilliams, Florence, AZ (US)

(21) Appl. No.: 17/616,305

(22) PCT Filed: Jun. 5, 2020
(86) PCT No.: PCT/US2020/036262

§ 371 Date: Dec. 3, 2021

Related U.S. Application Data
(60) Provisional application No. 62/857,915, filed on Jun. 6, 2019.

(51) Int. Cl. G01N 33/00

(52) U.S. Cl. CPC G01N 33/0098

(57) ABSTRACT

Provided is a proficiency testing material, and in more particular aspects, a cannabis proficiency testing material comprising: a plant substrate; and at least one chemical and/or microbial contaminant, wherein the at least one chemical and/or microbial contaminant is chemically or physically associated with the plant substrate. Methods of preparing the proficiency testing material and kits including the same are also provided.

### CLAIM OF PRIORITY

The present application claims the benefit of priority to U.S. Provisional Application Ser. No. 62/857,915, filed on Jun. 6, 2019, the contents of which is hereby incorporated herein by reference in its entirety.

### BACKGROUND

Proficiency testing (PT), or external quality assessment (EQA), is part of a quality system that provides external assessment and validation that laboratory performance meets certain standards and requirements. PT, for example, measures the ability of a laboratory to analyze specimens of unknown values and obtain accurate results. Examples of laboratories, and services provided by such laboratories, that benefit from proficiency testing include analytical laboratories and clinical laboratories, or any testing laboratory requiring regulatory oversight, for example, laboratories providing services to monitor, for example, medicaments, foodstuffs, and/or consumables for either human or non-human use. Included among such are tobacco and/or inhalable products. An emerging market requiring oversight and the benefits of proficiency testing is the cannabis market, both for medical and for recreational use.

A proficiency test is a method that may be used to validate processes involved in a particular measurement. The reference value of an artifact is not known by the participating laboratory at the time of its measurement (test). In a well-designed proficiency test, the reference value for the artifact should be principally determined by a competent laboratory with appropriate traceability to the International System of Units (SI). The reference laboratory should also have demonstrated competency though key comparisons, interlaboratory comparisons, or proficiency tests appropriate to validate its measurement capability. It is also preferable that the reference laboratory has had its competency independently assessed through the process of laboratory accreditation. Lastly, in order to appropriately validate the measurement capability of the laboratory participating in the proficiency test, the uncertainty assigned to the artifact by the reference laboratory should be sufficiently smaller than the expanded uncertainty reported by the participating laboratory.

Cannabis, which includes both marijuana and hemp, is a Schedule I controlled substance, placing it on the same list as heroin, LSD, MDMA and other illicit drugs deemed to have high abuse potential without an accepted medical use in the United States according to the Controlled Substances Act (CSA). However, more than half of the states in the United States have legalized medical cannabis, and several states, such as Colorado, Washington, California, Massachusetts, and Oregon, among others, have made allowances for recreational use of marijuana. This listing of cannabis as a Schedule I controlled substance precludes the U.S. Food and Drug Administration (FDA) from its regulation and oversight. This presents a major problem for state agencies and laboratories involved with regulatory oversight and public health monitoring of cannabis safety.

Beyond the matter of assessing cannabis potency is the equally important matter of ensuring cannabis for medical and recreational use is free of hazardous chemical and microbial contaminants. Cannabis is known to have a proclivity for concentrating dangerous heavy metals, so much so that industrial hemp has been used for phytoremediation of heavy metal polluted soils, and a study in California by the University of California at Davis in 2017 indicated that potentially dangerous microbes were detected on 90% of cannabis samples purchased from 20 different Northern California growers and dispensaries. Furthermore, over a period of 16 weeks in 2015, 16 cannabis product-related recalls in Colorado were issued, which included recalls for the presence of pesticides banned for use in Colorado. However, no single federal guideline exists, and individual states are beginning to develop regulations.

In view of the need for regulatory oversight of the cannabis industry, these state agencies and laboratories are tasked with monitoring labs that perform quality control testing of cannabis products. Proficiency testing (PT) and/or accreditation of cannabis testing labs is one of the manners in which regulatory oversight is accomplished. However, one problem stemming from current US policy is the federal ban on transporting Schedule I substances across state lines. This means that laboratory reference materials used, for example, in proficiency testing to validate testing protocols, conduct quality control checks, and to assure testing proficiency are required to come from in-state if the laboratory reference materials include controlled substances in non-exempt formulations. One example is PT performed in the state of Colorado by the Colorado Department of Public Health and Environment (CDPHE) for evaluating and certifying commercial cannabis-testing laboratories in Colorado. However, samples for PT used in CDPHE-developed protocols were derived from actual marijuana, thus preventing use in other states due to interstate transport restrictions for controlled substances.

Important to effective proficiency testing is use of an unknown sample, provided by a proficiency testing service, having characteristics that effectively mimic the general physical characteristics of known samples typically analyzed by the laboratory participating in the proficiency test. An unknown sample provided as part of a proficiency test that differs significantly in characteristics from typical samples provided to an analytical laboratory for testing can potentially lead to bias in the testing, created by knowledge of the sample being an unknown sample and being part of the proficiency test, as compared to typical samples analyzed by the laboratory.

As such, there is a need for improved materials and samples, and methods of using the same, suitable for PT of laboratories involved with monitoring of, for example, plant products for medical and/or recreational use, such as laboratories involved in monitoring of quality and/or safety of cannabis, that avoid the shortcomings of current products and methods available.

### SUMMARY OF THE INVENTION

Provided by the present inventive concept are materials and methods for proficiency testing of cannabis that can be distributed to any cannabis testing laboratory without the need of a DEA license.

In an aspect of the inventive concept, provided is a proficiency testing composition including: a plant substrate; and at least one chemical and/or microbial contaminant connected with the plant substrate.

In another aspect of the inventive concept, provided is a method for preparing a proficiency testing composition including: soaking an amount of a plant substrate material having a predetermined weight in a solution including at least one chemical or microbial contaminant of a predetermined concentration; and drying the plant substrate material on a non-absorbing surface, wherein the plant substrate material has a similar weight after drying to the predetermined weight of the plant substrate material prior to soaking.

In another aspect of the inventive concept, provided is a kit for proficiency testing including a proficiency testing composition including: a plant substrate; and at least one chemical or microbial contaminant incorporated into the plant substrate.

In another aspect of the inventive concept, provided is a method of proficiency testing including: providing a laboratory participating in a proficiency test the cannabis proficiency testing material of the inventive concept for testing by the laboratory; and analyzing test results obtained by the laboratory to determine or whether testing performed by the laboratory meets standards set by a regulating or governing organization administering the proficiency test.

In another aspect of the inventive concept, provided is a proficiency testing material including: a plant substrate; and at least one chemical and/or microbial contaminant, wherein the at least one chemical and/or microbial contaminant is uniformly incorporated into the plant substrate.

In another aspect of the inventive concept, provided is a method of proficiency testing including: providing a laboratory participating in a proficiency test the proficiency testing material of the inventive concept for testing by the laboratory; and analyzing test results obtained by the laboratory to determine or whether testing performed by the laboratory meets standards set by a regulating or governing organization administering the proficiency test.

### BRIEF DESCRIPTION OF THE DRAWING

FIG. 1 illustrates photographs and scanning electron micrographs (SEMs) of various plant substrate candidates for preparing cannabis proficiency testing materials of the present inventive concept.

### DETAILED DESCRIPTION

The foregoing and other aspects of the present inventive concept will now be described in more detail with respect to other embodiments described herein. It should be appreciated that the inventive concept can be embodied in different forms and should not be construed as limited to the embodiments set forth herein. Rather, these embodiments are provided so that this disclosure will be thorough and complete, and will fully convey the scope of the inventive concept to those skilled in the art.

The terminology used in the description of the inventive concept herein is for the purpose of describing particular embodiments only and is not intended to be limiting of the inventive concept. As used in the description of the inventive concept and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this inventive concept belongs.

### 1. Proficiency Testing Materials

The inventive concept is a product that can be used as a proficiency testing (PT) sample for proficiency testing/external quality assessment (EQA) of laboratories that perform testing on products provided by, for example, but not limited to, the cannabis industry (both marijuana and hemp). Although referred to more particularly herein, in exemplary embodiments, for PT/EQA to assess and/or validate laboratories, and measurements made by such laboratories, monitoring products provided by the cannabis industry, the PT/EQA samples of the present inventive concept may be utilized in PT/EQA of laboratories involved in testing of, for example, any plant product or byproduct that may generally share physical characteristics, such as general physical appearance, leaf structure, color, morphology etc., with those of products and byproducts provided by the cannabis industry. The product may contain at least one chemical and/or microbial contaminant, which is/are connected with, including, contained, applied, coated, embedded, and/or incorporated in a plant substrate, at known concentrations. Thus, the at least one chemical and/or microbial contaminant may be chemically or physically associated with the substrate. The chemical and/or microbial contaminant may be any contaminant or pollutant that may need monitoring for assurance of quality and/or safety of the product or byproduct for, e.g., human and/or animal ingestion, consumption, inhalation, i.e., use. In some embodiments, the monitoring performed by a testing or diagnostic laboratory may be for assessing the presence of a contaminant or pollutant in the environment, such, uptake of contaminants/pollutants by, for example, plants, which include cannabis/marijuana plants, as a result of pollution produced by and contamination as a result of human industrial activity, for example, but not limited to, production of steel, paper, coal, energy production, construction, pollution resulting from travel/transportation, waste disposal, etc. In some embodiments, the monitoring by a testing or diagnostic laboratory may be for assessing the presence of contaminants, and the like, present in the plant, such as cannabis/marijuana plants, resulting from intentional tampering or adulteration by an individual(s). For example, the substances added from tampering with or adulteration of, e.g., “cutting” cannabis/marijuana products/byproducts, may include licit or illicit substances that may modify/modulate/alter the effects and/or appearance of cannabis/marijuana products/byproducts consumed by the user of the cannabis/marijuana product/byproduct, and that may be considered undesirable for consumption by and/or the safety of the user, and/or considered undesirable for consumption or safety by regulating organizations of the cannabis/marijuana product/byproduct. Potential adulterants of cannabis/marijuana products/byproducts may include, e.g., phytocannabinoids, synthetic cannabinoids, residual solvents, microorganisms, pesticides, heavy metals and mycotoxins. Examples of licit and illicit adulterants of marijuana that may be considered undesirable include, but are not limited to, methamphetamine, ketamine, fentanyl, cocaine, laundry detergent, phencyclidine (PCP), LSD, formaldehyde, perfumes, glass, and/or heroin, and other synthetic psychoactive substances, such as synthetic cannabinoids and cathinones. In some embodiments, contaminants may also include any poison, e.g., a heavy metal, such as, but not limited to, arsenic, cadmium, and/or lead, or pesticide, such as, but not limited to, carbofuran, and/or myclobutanil, as would be appreciated by one of skill in the art.

In some embodiments, the plant substrate may be the buds, flowers, stems, and/or leaves of a plant that is structurally and/or morphologically similar to cannabis. In some embodiments, the plant substrate is not a controlled substance, i.e., does not require a DEA license for sale, for example, from a vendor in one state to a recipient in another state, and thus can be distributed to any laboratory. In other embodiments, the plant substrate does not contain cannabinoids. In other embodiments, the plant substrate does not have a strong, distinct odor. In other embodiments, the plant substrate is widely available as organic/pesticide free due to its intended use for pets, for example, catnip. In other embodiments, the plant substrate has morphological similarities to cannabis based on general appearances and SEM images. In some embodiments, the plant material may be derived from, for example, Turnera diffusa (damiana), Nepeta cataria (catnip), Althaea cannabina and Althaea officinalis (marsh-mallow), Scutellaria (skullcap), Mentha (mint), members of the Cleomaceae family (spider plants), Hibiscus cannabinus (kenaf) and/or Humulus lupulus (hops). In an embodiment, the plant substrate may be catnip. In another embodiment, the plant substrate may be marsh-mallow.

In some embodiments, the contaminant connected, contained, applied, coated, embedded, and/or incorporated in the plant substrate is uniform or homogeneous, so that any particular amount of the material may be analyzed, for example, about 50 mg to about 100 mg plant material, to match the normal analytical method of a particular laboratory participating in a proficiency test.

The chemical or microbial contaminant, which is connected, contained, applied, coated, embedded, and/or incorporated into the plant substrate is not particularly limited. However, the chemical or microbial contaminant, in some embodiments, is a contaminant that may be found in cannabis and present a health and/or safety hazard to a user of a cannabis product.

Further examples of chemical contaminants include, in some embodiments, heavy metals, such as, but are not limited to, Arsenic (As), Cadmium, (Cd), Cobalt (Co), Chromium (Cr), Copper (Cu), Mercury (Hg), Manganese (Mn), Nickel (Ni), Lead (Pb), Thallium (Tl), and/or Zinc (Zn). In other embodiments, the chemical contaminant may be a residual solvent, examples of which include, but are not limited to, n-hexane, n-heptane, n-pentane, 2-propanol, ethanol, acetone, acetonitrile, tetrahydrofuran, toluene, chloroform, carbon tetrachloride, benzene, m-xylene, o-xylene, p-xylene and/or methanol. In other embodiments, the chemical contaminant may be a pesticide, further examples of which include, but are not limited to, abamectin, acephate, aldicarb, acequinocyl, azoxystrobin, bifenazate, bifenthrin, boscalid, brodifacoum, bromodiolone, captan, carbofuran, carbaryl, chlorantraniliprole, chlordane, chlorfenapyr, chlorpyrifos, coumaphos, cyfluthrin, cypermethrin, daminozide, diazinon, dichlorvos, dimethoate, dimethomorph, difenacoum, difethialone, DDVP (Dichlorvos), ethoprop(hos), etofenprox, etoxazole, fenhexamid, fenoxycarb, fipronil, flonicamid, fludioxonil, imazilil, imidacloprid, malathion, methyl parathion, methiocarb, methomyl, mevinphos, metalaxyl, myclobutanil, naled, paclobutrazol, permethrin, piperonyl butoxide, PCNB, propiconazole, propoxur, pyrethrin, spinetoram, spinosad, spiromesifen, spirotetramat, spiroxamine, tebuconazole, thiacloprid, thiamethoxam, and/or trifloxystrobin.

Examples of microbial contaminants may include, for example, bacteria, fungi, mold, protozoa, yeast, and/or toxins or byproducts from any one thereof. Bacteria include pathogenic bacteria, for example, Escherichia coli, Klebsiella, Salmonella, and Shigella, among others. Fungi and toxins or byproducts thereof include, for example, Alternaria, Aspergillus, Fusarium, Paecilomyces, Penicillium, Stachybotrys, and mycotoxins, such as aflatoxin, citrinin, fumonisins, ochratoxin A, patulin, trichothecenes, zearalenone, and ergot alkaloids, such as ergotamine.

### 2. Methods of Preparation

In some embodiments, the proficiency testing material may include a predetermined amount or concentration of a chemical or microbial contaminant uniformly contained, embedded and/or incorporated in the plant substrate. Any method as would be appreciated by one of skill in the art may be used to prepare the proficiency testing material, so long as the method provides a material which includes the chemical or microbial contaminant at a known, predetermined concentration that is uniformly contained, embedded and/or incorporated into the plant substrate. Uniform distribution of the chemical and/or microbial contaminant in the plant substrate may be useful in the proficiency testing material if, for example, a prepared batch of the proficiency testing material is divided into multiple test samples. uniform distribution of the chemical and/or microbial contaminant provides/assures there is a consistent amount of the chemical or microbial contaminant in each of the multiple test samples.

The proficiency testing material may be, for example, prepared by combining a plant substrate and a solution, e.g., by soaking the plant substrate in, adding the plant substrate to, or exposing the plant substrate to the solution. The solution, such as an aqueous solution, may be prepared containing, for example, metal salts and/or one or more pesticides, at an appropriate concentration to prepare a proficiency testing material containing the desired final product specifications. For example, a plant substrate, such as catnip buds or marshmallow leaf, may be soaked in an aqueous metal salt or pesticide solution for a suitable period of time, e.g., about 12 hours, about 16 hours, about 20 hours or about 24 hours, to ensure substantially complete adsorption of the desired contaminant in solution by the plant substrate, using, for example, about 3 mL of solution for each gram of plant substrate. The material may then be removed from the solution and dried on a non-absorbing surface, such as aluminum foil. The material is dried until the final weight is near the starting weight.

In other embodiments, the proficiency testing material may be prepared by spraying the plant substrate with a solution, such as an aqueous solution, prepared containing, for example, one or more metal salts, one or more pesticides, and/or microbial contaminants contained at an appropriate concentration in the solution to prepare a proficiency testing material with the desired final product specifications. The plant substrate may be sprayed with the solution more than once in order to achieve the desired product specifications. In some embodiments, the solution may be sprayed onto the plant substrate using a nebulizer. The plant substrate may be sprayed with a known amount of the solution that is adsorbed by the plant substrate that contains an amount of a metal salt(s), pesticide, and/or a microbial contaminant, such as bacterial spores, suitable to prepare a proficiency testing material having the desired final product specifications.

### 3. Methods of Use

The proficiency testing materials as described herein may be used in any type of proficiency testing scheme, for example, but not limited to, a scheme designed for monitoring and/or assessing participating testing facilities, such as cannabis testing facilities, e.g., testing and/or diagnostic laboratories that analyze plant products/byproducts, such as cannabis products/byproducts for, e.g., contaminants/safety of, for example, the cannabis products/byproducts for human or animal use/consumption. Types of proficiency tests include: blind proficiency tests, where the testing facility does not know it is being tested; external proficiency tests, i.e., a proficiency test obtained from and reported to a proficiency test provider external to the quality assurance system of the participant; internal proficiency tests, i.e., a proficiency test designed and administered within the quality assurance system of the participant; and open or declared proficiency tests, i.e., a proficiency test in which participants know they are being tested. Furthermore, in some embodiments, the proficiency testing material may be provided as part of a proficiency testing kit available to a testing or diagnostic laboratory, such as a cannabis testing or diagnostic laboratory participating in a proficiency test.

Having described various aspects of the present inventive concept, the same will be explained in further detail in the following examples, which are included herein for illustration purposes only, and which are not intended to be limiting to the invention.

### Examples

### 1. Preparation

An aqueous solution is prepared by adding metal salts and/or pesticides at appropriate concentrations for the desired final product specifications. Catnip buds are soaked in the solution for 16 hours. 3 mL solution is used for each 1 g of plant material. This ensures almost complete absorption of the solution by the plant material. The plant material is then removed from the container and placed on aluminum foil (or any other non-absorbing surface) until dry. The material is dried until the final weight is near the starting weight.

Water uptake and analyte absorption efficiency factor are experimentally determined for each batch of plant material. Target analyte concentration (μg/g)=initial analyte concentration (μg/g)+stock concentration (μg/mL)×water uptake (mL/g)×analyte absorption efficiency factor. For example, one batch of PT containing catnip with 1 μg/g carbofuran, 0.5 μg/g myclobutanil, and 2 μg/g lead and arsenic was prepared by soaking 40 g of plant material in 120 mL of an aqueous solution containing lead (0.500 μg/mL), arsenic (0.500), myclobutanil (0.150 μg/mL) and carbofuran (0.260 μg/mL) overnight.

Current analytes included in the PT material were selected based on the cannabis testing requirements in California. Additional components from the same test categories (pesticides and/or metals) can be added using the same procedure. The final product can also be inoculated with microbial impurities and/or mycotoxins after the metals and pesticides are incorporated.

### 2. Characteristics

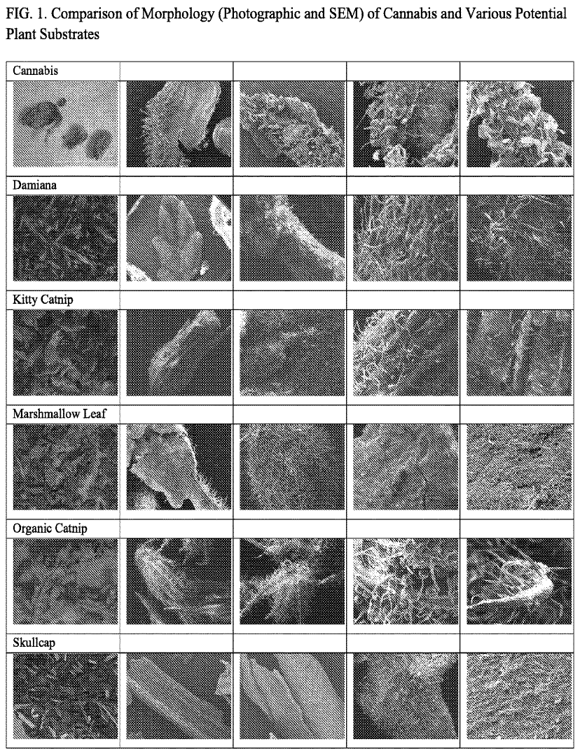

FIG. 1 depicts photographic and SEM characterization of the morphologies of cannabis and various potential plant substrates for the proficiency testing material. The plant substrates used in the preparation of proficiency testing materials are catnip (Nepeta cataria) buds and marshmallow (Althaea cannabina). Catnip buds as a plant substrate can provide the following advantages:
it is not a controlled substance (federally or by any state)
does not contain potentially interfering cannabinoids
does not have a strong distinct odor
contains similar structural components to cannabis bud/flowers (as opposed to many available plant products, such as teas, that contain mainly leaves)
is widely available as organic/pesticide free due to intended use for pets
it has morphological similarities to cannabis based on SEM images (See, FIG. 1)
tests with metals incorporation demonstrated adequate homogeneity (See, Tables 1 and 2)

Other non-controlled plants could be used as well. The main desired characteristic for any plant material used in cannabis testing would be that it contains flower/buds to mimic authentic cannabis samples and has adequate homogeneity of absorption. Candidate plant substrates include those depicted in FIG. 1.

Evaluation of the incorporation of various heavy metals into the plant substrate proficiency testing material is shown by the results shown in Tables 1 and 2. Table 1 shows the incorporation of Pb and the natural abundances of As, Cd, and Hg into two different sources of catnip and marshmallow leaf. Table 2 shows the incorporation of As and Pb into catnip. These tests with heavy metal incorporation demonstrated adequate homogeneity.

The foregoing is illustrative of the present inventive concept and is not to be construed as limiting thereof. The invention is defined by the following claims, with equivalents of the claims to be included therein.

TABLE 1
Metal incorporation results for lead in different plant substrates.

| | As | Cd | Hg | Pb | | |
|---|---|---|---|---|---|---|
| 12171-75-01.1 | 33.0 | 205 | 22.7 | 1473 | Marshmallow Leaf | As and Cd show the |
| 12171-75-01.2 | 28.3 | 201 | <DL | 1442 | Marshmallow Leaf | sample is fairly |
| 12171-75-01.3 | 30.6 | 188 | <DL | 1532 | Marshmallow Leaf | uniform. |
| 12171-75-01.4 | 30.5 | 192 | <DL | 1378 | Marshmallow Leaf | |
| 12171-75-01.5 | 32.2 | 207 | <DL | 1778 | Marshmallow Leaf | |
| Average | 30.9 | 198 | 22.7 | 1521 | | |
| Standard Deviation | 1.8 | 8.4 | n/a | 154 | 10% | |
| 12171-76-01.1 | 31.6 | 155 | <DL | 1398 | Organic Catnip | As and Cd show high |
| 12171-76-01.2 | 24.7 | 46.4 | <DL | 2358 | Organic Catnip | variability in sample. |
| 12171-76-01.3 | 25.1 | 44.5 | <DL | 2330 | Organic Catnip | |
| 12171-76-01.4 | <DL | 39.0 | <DL | 2315 | Organic Catnip | |
| 12171-76-01.5 | 34.2 | 43.3 | <DL | 2636 | Organic Catnip | |
| Average | 28.9 | 65.7 | <DL | 2207 | | |
| Standard Deviation | 13.6 | 50.2 | n/a | 471 | 21% | |
| 12171-79-01.1 | 68.6 | <DL | <DL | 3041 | Kitty Catnip | As shows sample has |
| 12171-79-01.2 | 78.4 | <DL | <DL | 2940 | Kitty Catnip | some uniformity |
| 12171-79-01.3 | 62.6 | <DL | <DL | 2376 | Kitty Catnip | issues. If you ever |
| 12171-79-01.4 | 85.4 | <DL | <DL | 3589 | Kitty Catnip | need to spike Cd, |
| 12171-79-01.5 | 76.9 | <DL | <DL | 3280 | Kitty Catnip | this would be the |
| Average | 74.4 | <DL | <DL | 3045 | | |
| Standard Deviation | 8.9 | n/a | n/a | 450 | 15% | |
| | | | | | | Pb avg without 2376 |
| | | | | | | 3212 |
| | | | | | | 288.8707148 |
| | | | | | | 0.089926489 |

^{a}Calculated as: instrument result (ng/mL)
*digestion volume (10 mL)/mass of sam [symbol]
DL for As, Cd, Hg, and Pb = 20 ng/g
Nominal mass for each sample was 50 mg.
Same digestion procedure as last time. [symbol]
indicates data missing or illegible when filed

TABLE 2
Metal incorporation results for arsenic and lead in catnip.

| | | As | % Recovery | Pb | % Recovery |
|---|---|---|---|---|---|
| | Blank | 0.009 | n/a | −0.016 | n/a |
| | 10 | 9.974 | 99.74 | 10.33 | 103.3 |
| | 20 | 19.76 | 98.8 | 20.21 | 101.05 |
| | 50 | 49.93 | 99.86 | 50.11 | 100.22 |
| | Check1 | 20.14 | 100.7 | 19.4 | 97 |
| | Blank1 | 0.022 | n/a | 0.017 | n/a |
| | CFS-1 | 17.5 | | 15.73 | |
| | CFS-2 | 16.61 | | 14.34 | |
| | CFS-3 | 19.55 | | 15.1 | |
| | CFS-4 | 17.51 | | 14.08 | |
| | CFS-5 | 18.62 | | 14.03 | |
| | Check2 | 20.79 | 103.95 | 19.23 | 96.15 |
| | Blank2 | 0.027 | n/a | 0.015 | n/a |
| Weight, g | | As | μg/g | Pb | μg/g |
| 0.0997 | CFS-1 | 17.5 | 1.76 | 15.73 | 1.58 |
| 0.0953 | CFS-2 | 16.61 | 1.74 | 14.34 | 1.50 |
| 0.0996 | CFS-3 | 19.55 | 1.96 | 15.1 | 1.52 |
| 0.1003 | CFS-4 | 17.51 | 1.75 | 14.08 | 1.40 |
| 0.0986 | CFS-5 | 18.62 | 1.89 | 14.03 | 1.42 |
| | | Ave. | 1.82 | | 1.49 |
| | | STDEV | 0.101 | | 0.0714 |

1. A cannabis proficiency testing material comprising:
a plant substrate; and
at least one chemical and/or microbial contaminant,
wherein the at least one chemical and/or microbial contaminant is incorporated into the plant substrate.

2. The cannabis proficiency testing material of claim 1, wherein the at least one chemical and/or microbial contaminant is a heavy metal.

3. The cannabis proficiency testing material of claim 2, wherein the heavy metal is selected from the group consisting of Arsenic (As), Cadmium, (Cd), Cobalt (Co), Chromium (Cr), Copper (Cu), Mercury (Hg), Manganese (Mn), Nickel (Ni), Lead (Pb), Thallium (Tl) and Zinc (Zn).

4. The cannabis proficiency testing material of claim 1, wherein the at least one chemical and/or microbial contaminant is a pesticide.

5. The cannabis proficiency testing material of claim 4, wherein the pesticide is selected from the group consisting of abamectin, acephate, aldicarb, acequinocyl, azoxystrobin, bifenazate, bifenthrin, boscalid, brodifacoum, bromodiolone, captan, carbofuran, carbaryl, chlorantraniliprole, chlordane, chlorfenapyr, chlorpyrifos, coumaphos, cyfluthrin, cypermethrin, daminozide, diazinon, dichlorvos, dimethoate, dimethomorph, difenacoum, difethialone, DDVP (Dichlorvos), ethoprop(hos), etofenprox, etoxazole, fenhexamid, fenoxycarb, fipronil, flonicamid, fludioxonil, imazilil, imidacloprid, malathion, methyl parathion, methiocarb, methomyl, mevinphos, metalaxyl, myclobutanil, naled, paclobutrazol, permethrin, piperonyl butoxide, PCNB, propiconazole, propoxur, pyrethrin, spinetoram, spinosad, spiromesifen, spirotetramat, spiroxamine, tebuconazole, thiacloprid, thiamethoxam, and trifloxystrobin.

6. The cannabis proficiency testing material of claim 1, wherein the at least one chemical and/or microbial contaminant is a residual solvent selected from the group consisting of n-hexane, n-heptane, n-pentane, 2-propanol, ethanol, acetone, acetonitrile, tetrahydrofuran, toluene, chloroform, carbon tetrachloride, benzene, m-xylene, o-xylene, p-xylene and methanol.

7. The cannabis proficiency testing material of claim 1, wherein the at least one chemical and/or microbial contaminant is a microbial contaminant.

8. The cannabis proficiency testing material of claim 7, wherein the microbial contaminant is a bacteria, fungi, mold, protozoa, yeast, or a toxin or byproduct of any one thereof.

9. The cannabis proficiency testing material of claim 1, wherein the plant substrate is a bud, flower, stem and/or leaf of a plant that is structurally similar to cannabis.

10. The cannabis proficiency testing material of claim 1, wherein the plant substrate is selected from the group consisting of Turnera diffusa (damiana), Nepeta cataria (catnip), Althaea cannabina and Althaea officinalis (marsh-mallow), Scutellaria (skullcap), Mentha (mint), members of the Cleomaceae family (spider plants), Hibiscus cannabinus (kenaf) and/or Humulus lupulus (hops).

11. A method of preparing a cannabis proficiency testing material comprising:
combining a plant substrate and an aqueous solution comprising at least one chemical and/or microbial contaminant; and
drying the plant substrate.

12. The method of claim 11, wherein the at least one chemical and/or microbial contaminant is a heavy metal.

13. The method of claim 12, wherein the heavy metal is selected from the group consisting of Arsenic (As), Cadmium, (Cd), Cobalt (Co), Chromium (Cr), Copper (Cu), Mercury (Hg), Manganese (Mn), Nickel (Ni), Lead (Pb), Thallium (Tl) and Zinc (Zn).

14. The method of claim 11, wherein the at least one chemical and/or microbial contaminant is a pesticide.

15. The method of claim 14, wherein the pesticide is selected from the group consisting of abamectin, acephate, aldicarb, acequinocyl, azoxystrobin, bifenazate, bifenthrin, boscalid, brodifacoum, bromodiolone, captan, carbofuran, carbaryl, chlorantraniliprole, chlordane, chlorfenapyr, chlorpyrifos, coumaphos, cyfluthrin, cypermethrin, daminozide, diazinon, dichlorvos, dimethoate, dimethomorph, difenacoum, difethialone, DDVP (Dichlorvos), ethoprop(hos), etofenprox, etoxazole, fenhexamid, fenoxycarb, fipronil, flonicamid, fludioxonil, imazilil, imidacloprid, malathion, methyl parathion, methiocarb, methomyl, mevinphos, metalaxyl, myclobutanil, naled, paclobutrazol, permethrin, piperonyl butoxide, PCNB, propiconazole, propoxur, pyrethrin, spinetoram, spinosad, spiromesifen, spirotetramat, spiroxamine, tebuconazole, thiacloprid, thiamethoxam, and trifloxystrobin.

16. The method of claim 11, wherein the at least one chemical and/or microbial contaminant is a residual solvent selected from the group consisting of n-hexane, n-heptane, n-pentane, 2-propanol, ethanol, acetone, acetonitrile, tetrahydrofuran, toluene, chloroform, carbon tetrachloride, benzene, m-xylene, o-xylene, p-xylene and methanol.

17. The method of claim 11, wherein the at least one chemical and/or microbial contaminant is a microbial contaminant.

18. The method of claim 17, wherein the microbial contaminant is a bacteria, fungi, mold, protozoa, yeast, or a toxin or byproduct of any one thereof.

19. The method of claim 11, wherein the plant substrate is a bud, flower, stem and/or leaf of a plant that is structurally similar to cannabis.

20. The method of claim 11, wherein the plant substrate is selected from the group consisting of Turnera diffusa (damiana), Nepeta cataria (catnip), Althaea cannabina and Althaea officinalis (marsh-mallow), Scutellaria (skullcap), Mentha (mint), members of the Cleomaceae family (spider plants), Hibiscus cannabinus (kenaf) and/or Humulus lupulus (hops).

21. A kit for proficiency testing of a cannabis diagnostic laboratory comprising a cannabis proficiency testing material comprising the cannabis proficiency testing material of claim 1.

22. A proficiency testing material comprising:
a plant substrate; and
at least one chemical and/or microbial contaminant,
wherein the at least one chemical and/or microbial contaminant is incorporated into the plant substrate.

23. A method of proficiency testing comprising:
analyzing test results obtained by a laboratory participating in a proficiency test for the cannabis proficiency testing material of claim 1 to determine whether testing performed by the laboratory meets standards set by a regulating or governing organization administering the proficiency test.

24. A method of proficiency testing comprising:
analyzing test results obtained by a laboratory participating in a proficiency test of the proficiency testing material of claim 22 to determine whether testing performed by the laboratory meets standards set by a regulating or governing organization administering the proficiency test.

25. The method of claim 23, wherein the laboratory participating in the proficiency test monitors for a presence of chemical and/or microbial contaminants in a cannabis product or byproduct that result from tampering and/or adulteration of the cannabis product or byproduct by an individual(s).
